(19) United States
(12) Patent Application Publication
(10) Pub. No.: US 2026/0348841 A1
(43) Pub. Date: Nov. 5, 2026

(54) ABSORPTION CELL FOR GAS CHARACTERIZATION

(71) Applicant: Clean Connect AI, Inc., Windsor, CO (US)

(72) Inventors: Emily Lynn Wilson, Edgewater, MD (US); Remington Engelhard, Windsor, CO (US); David A. Conley, Windsor, CO (US)

(21) Appl. No.: 19/665,162

(22) Filed: May 1, 2026

Related U.S. Application Data
(60) Provisional application No. 63/798,792, filed on May 2, 2025.

(51) Int. Cl. G01N 21/31; G01N 33/00

(52) U.S. Cl. CPC G01N 21/31; G01N 33/0027; G01N 2201/06113; G01N 2201/0636; G01N 2201/08

(57) ABSTRACT

Various embodiments of the present technology relate to gas composition characterization. Some embodiments comprise a gas characterization testing system that comprises a waveguide absorption cell, processing circuitry, a laser, and a photodetector. The waveguide absorption cell receives a gas sample. The waveguide absorption cell comprises a cell body that defines an optical path through the gas sample. The laser generates an optical beam and directs the optical beam through the optical path of the waveguide absorption cell. The optical beam interacts with the gas sample. The photodetector detects the optical beam after its interaction with the gas sample and generates a signal based on the detection. The processing circuitry determines the composition of the gas sample based on the signal.

### CROSS-REFERENCE TO RELATED APPLICATIONS

This U.S. Patent Application claims the benefit of and priority to U.S. Provisional Patent Application 63/798,792 titled, “ABSORPTION CELL FOR GAS CHARACTERIZATION” which was filed on May 2, 2025. U.S. Provisional Patent Application 63/798,792 is hereby incorporated by reference in its entirety into this U.S. Patent Application.

### TECHNICAL FIELD

Various embodiments of the present technology relate to gas characterization, and more specifically, to an absorption cell for characterizing gas compositions.

### BACKGROUND

Hydrocarbon extraction systems comprise machinery and equipment configured to extract petroleum, natural gas, and other chemicals for use in energy generation, heating, and chemical production applications. Hydrocarbon extraction systems comprise extraction equipment, transfer equipment, and storage equipment. The extraction equipment removes hydrocarbons from subterranean reservoirs. Examples of extraction equipment include drilling rigs and hydraulic fracturing devices. The transfer equipment transports the extracted hydrocarbons between different geographic locations. Examples of transfer equipment include pipelines and tanker trucks. The storage equipment stores hydrocarbons. Examples of storage equipment include bullet tanks and storage vessels.

When extracting, storing, or transferring natural gas or other gaseous hydrocarbons, pressure can accumulate in the hydrocarbon extraction, transfer, and storage equipment. To respond to excess natural gas, the hydrocarbon extraction system burns off the excess natural gas. During combustion, the natural gas reacts with oxygen and is converted to carbon dioxide and water which is expelled into the atmosphere. This reaction does not run to completion due to imperfect reaction conditions and unreacted natural gas as well as unwanted side-reaction products are also expelled into the atmosphere. Environmental regulations dictate that the proportion of unreacted natural gas in combustion exhaust cannot exceed a threshold, typically 2%. The combustion systems that burn natural gas are designed to operate within the threshold set by these regulations. However, the combustion efficiency of these systems degrades over time due to deterioration.

To ensure the combustion systems burn natural gas within the limit set by environmental regulations, human operators manually test the chemical composition of the exhaust gas. Manually testing burning exhaust is dangerous to human operators. Moreover, the manual testing is not performed under normal operating conditions (e.g., when burning off excess natural gas). Unfortunately, conventional hydrocarbon processing systems do not efficiently determine the chemical composition of natural gas exhaust. Moreover, the hydrocarbon processing systems do not effectively adjust gas/air ratios during combustion based on exhaust gas composition.

### Overview

This Overview is provided to introduce a selection of concepts in a simplified form that are further described below in the Technical Description. This summary is not intended to identify key features or essential features of the claimed subject matter, nor is it intended to be used as an aid in determining the scope of the claimed subject matter.

Various embodiments of the present technology relate to solutions for gas characterization. Some embodiments comprise a gas characterization system. The gas characterization system comprises a waveguide absorption cell, processing circuitry, a laser, and a photodetector. The waveguide absorption cell receives a gas sample. The waveguide absorption cell comprises a cell body that defines an optical path through the gas sample. The laser generates an optical beam and directs the optical beam through the optical path of the waveguide absorption cell. The optical beam interacts with the gas sample. The photodetector detects the optical beam after its interaction with the gas sample and generates a signal based on the detection. The processing circuitry determines the composition of the gas sample based on the signal.

Some embodiments comprise a method. The method comprises passing a gas sample through a waveguide absorption cell. The waveguide absorption cell comprises a cell body that defines an optical path through the gas sample. The method further comprises generating an optical beam and directing the optical beam through the optical path of the waveguide absorption cell. The optical beam interacts with the gas sample. The method further comprises detecting the optical beam after its interaction with the gas sample and generating a signal based on the detection. The method further comprises determining the composition of the gas sample based on the signal.

Some embodiments comprise a waveguide absorption cell. The waveguide absorption cell comprises a cell body, one or more optical windows, and at least one gas port. The cell body has one or more open features that form a folded optical path that increases an interaction between a gas sample and an optical beam. The total length of the folded optical path is greater than the largest dimension of the cell body. The one or more optical windows allow the optical beam to pass through the folded optical path. The at least one gas port allows the gas sample to flow through the folded optical path.

### DESCRIPTION OF THE DRAWINGS

Many aspects of the disclosure can be better understood with reference to the following drawings. The components in the drawings are not necessarily drawn to scale. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 illustrates an example of an operating environment for a gas composition testing system that characterizes the composition of a gas.

FIG. 2 illustrates an example operation of the gas composition testing system to characterize the composition of a gas.

FIG. 3A illustrates an example of a waveguide absorption cell in the gas composition testing system that characterizes the composition of a gas.

FIG. 3B illustrates another example of the waveguide absorption cell in the gas composition testing system that characterizes the composition of a gas.

FIG. 3C illustrates another example of the waveguide absorption cell in the gas composition testing system that characterizes the composition of a gas.

FIG. 4A illustrates an example operating configuration of the waveguide absorption cell in the gas composition testing system that characterizes the composition of a gas.

FIG. 4B illustrates another example operating configuration of the waveguide absorption cell in the gas composition testing system that characterizes the composition of a gas.

FIG. 5 illustrates an example of another gas composition testing system that operates in direct absorption mode to characterize the composition of a gas.

FIG. 6 illustrates an example of the other gas composition testing system that operates in wavelength-modulation detection mode to characterize the composition of a gas.

FIG. 7 illustrates an example of a computing system that may be used in accordance with various embodiments of the present technology.

The drawings have not necessarily been drawn to scale. Similarly, some components or operations may not be separated into different blocks or combined into a single block for the purposes of discussion of some of the embodiments of the present technology. Moreover, while the technology is amendable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### TECHNICAL DESCRIPTION

The following description and associated figures teach the best mode of the invention. For the purpose of teaching inventive principles, some conventional aspects of the best mode may be simplified or omitted. The following claims specify the scope of the invention. Note that some aspects of the best mode may not fall within the scope of the invention as specified by the claims. Thus, those skilled in the art will appreciate variations from the best mode that fall within the scope of the invention. Those skilled in the art will appreciate that the features described below can be combined in various ways to form multiple variations of the invention. As a result, the invention is not limited to the specific examples described below, but only by the claims and their equivalents.

Natural gas combustion is the controlled burning of excess natural gas that is released during oil and gas production, processing, or refining when the gas cannot be economically captured, transported, or used. When natural gas is burned, the combustion reaction (i.e., the oxidation of methane) theoretically produces carbon dioxide and water as reaction products. However, in real-world operating conditions, a portion of the natural gas will not react. This is due to imperfect reaction conditions, which prevent the reaction from going to completion. Natural gas is considered a pollutant and is a potent green-house gas. As such, environmental regulations dictate that the proportion of unreacted natural gas during combustion cannot exceed a threshold, typically 2%.

Conventional combustion systems that brn natural gas are designed to operate within the threshold set by these regulations. While these systems are designed to operate below the threshold, their combustion efficiency degrades over time. To ensure the combustion systems burn natural gas within the limit set by environmental regulations, human operators manually test the chemical composition of the exhaust gas. Manually testing the exhaust of combusted natural gas is a dangerous and time consuming process. Manual testing is also not performed under normal operating conditions. Unfortunately, conventional oil and gas production, processing, or refining systems do not efficiently determine the chemical composition of combusted natural gas exhaust. Moreover, the conventional oil and gas production, processing, or refining systems do not effectively adjust gas/air ratios during combustion based on exhaust gas composition.

To address the above described problems in conventional oil and gas production, processing, or refining systems, various embodiments of the present technology relate to a gas characterization system. The gas characterization system includes a waveguide absorption cell, a tunable optical source, a photodetector, and processing circuitry configured to analyze optical signals generated as light interacts with a gas sample. The system may operate in one or more detection modes, including direct absorption detection and Wavelength Modulation Spectroscopy (WMS), to enable accurate measurement of gas composition across a wide range of concentrations. By employing configurable optical paths, signal modulation and demodulation, and shared optical and electronic components, the gas characterization system provides robust, high-sensitivity gas measurements suitable for monitoring exhaust streams, combustion processes, and other industrial or environmental gas-producing systems. This provides an efficient and effective alternative to the manual testing procedures used in conventional oil and gas production, processing, or refining systems. Now referring to the Figures.

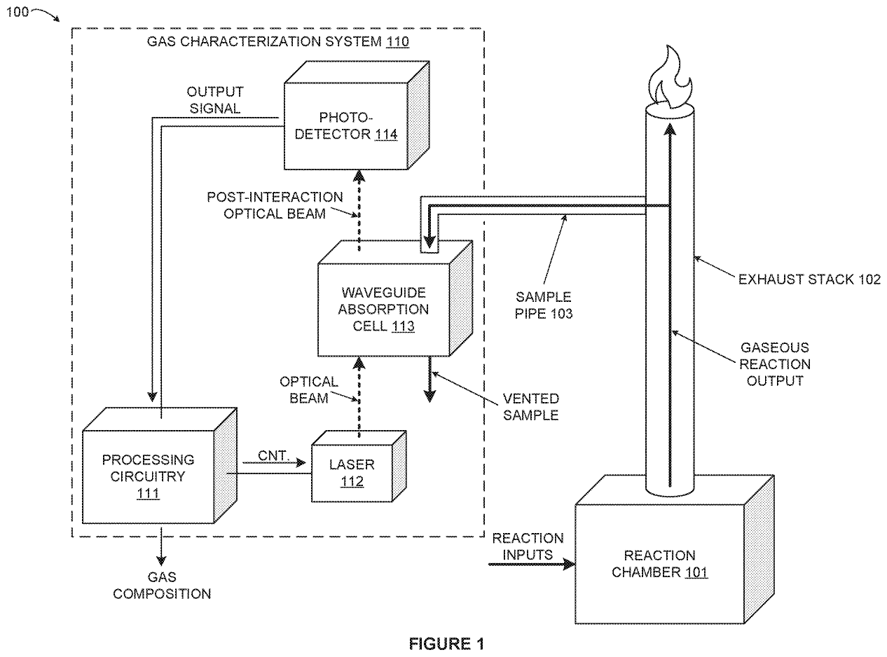

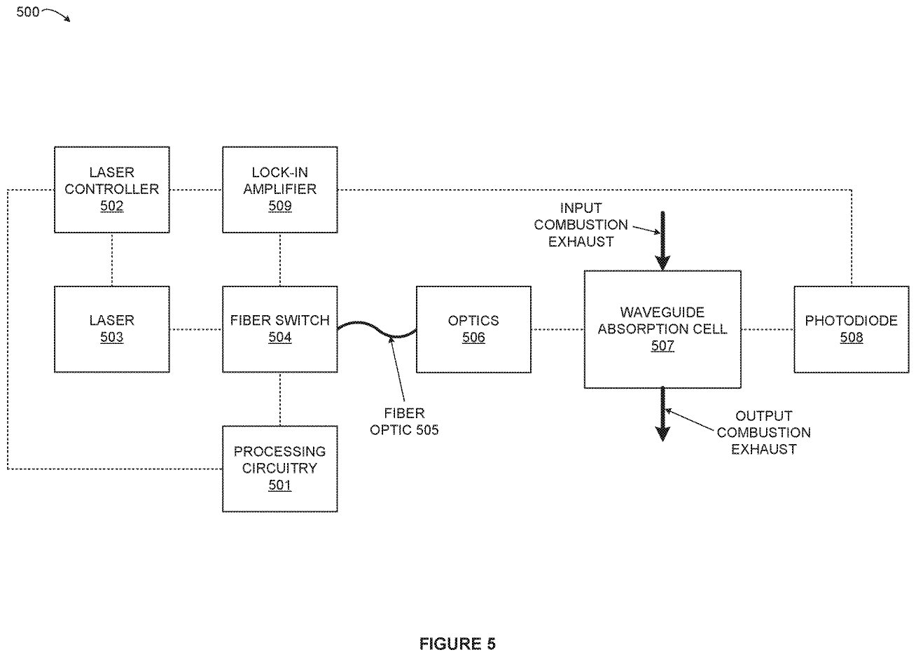

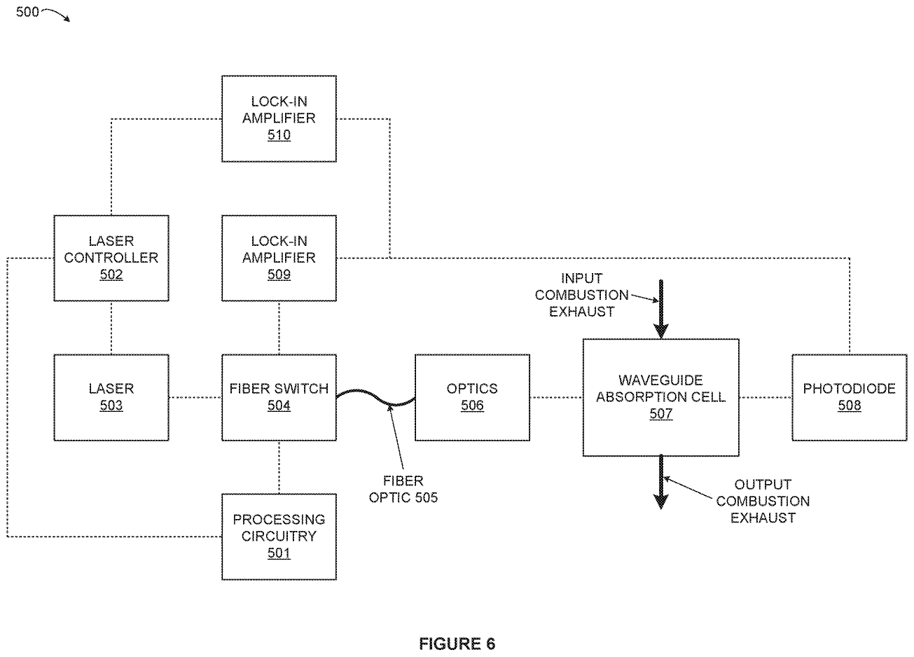

FIG. 1 illustrates an example of operating environment 100. Operating environment 100 may be representative of a hydrocarbon combustion, chemical manufacturing, energy production, material processing, mining, and/or another operating environment that utilizes a chemical reaction that generates a gaseous reaction product. Operating environment 100 comprises reaction chamber 101, exhaust stack 102, sample pipe 103, and gas characterization system 110. Gas characterization system 110 characterizes the composition of a gas. Gas characterization system 110 performs operations like determining gas composition, sampling gas, and controlling reaction inputs that drive gas creation based on the determined gas composition in operating environment 100. Gas characterization system 110 comprises an example of gas composition testing system 500 illustrated in FIGS. 5 and 6, however gas composition testing system 500 may differ. Gas characterization system 110 comprises processing circuitry 111, laser 112, waveguide absorption cell 113, and photodetector 114. Gas characterization system 110 typically comprises additional components like lock-in amplifiers, laser controllers, fiber switches, fiber optics, optics, and the like, however these additional components are omitted for clarity. In other examples, operating environment 100 and gas characterization system 110 may include fewer or additional components than those illustrated in FIG. 1. Likewise, the illustrated components of operating environment 100 and gas characterization system 110 may include fewer or additional components, assets, or connections than shown.

Various examples of system architecture and operation are described herein. In some examples, reaction chamber 101 receives reaction inputs (e.g., fuel and air) and initiates a chemical reaction (e.g., combustion). Operating environment 100 may include a reaction controller that controls the composition and flowrate of reaction inputs to reaction chamber 101, however the reaction controller is omitted for clarity. The reaction produces a gaseous reaction output (e.g., in the case of hydrocarbon combustion, carbon dioxide). The gaseous reaction output flows through exhaust stack 102 and is expelled into the atmosphere. In other examples, the gaseous reaction output may instead be captured and stored or may be delivered to another reaction chamber to be used as an input for another chemical reaction. Due to imperfect reaction conditions, a portion of the reaction inputs go unreacted and/or undesired reaction side products (e.g., products of incomplete combustion) may be produced. Exemplary imperfect reaction conditions include unfavorable reaction input ratios, reaction buffers, temperature and the like. For example, in the case of combusting natural gas, if the ratio of natural gas to air is too large (i.e., not enough oxygen to combust the natural gas), a threshold level (e.g., 2%) of natural gas will go unreacted. Likewise, if the ratio of natural gas to air is too small (i.e., too much available nitrogen), undesired side reactions may produce side reaction products like nitrous oxide.

Gas characterization system 110 collects a portion of the gaseous reaction output via sample pipe 103. The gas sample flows through sample pipe 103 into waveguide absorption cell 113. For example, gas characterization system 110 may be equipped with a vacuum pump, fan, or other device to create a pressure differential to drawn in a portion of the exhaust through sample pipe 103. The gas sample passes through waveguide absorption cell and is expelled into the atmosphere. Gas characterization system 110 may include one or more valves to control gas flowrate into and the pressure inside waveguide absorption cell 113. It should be appreciated that by reducing the pressure in waveguide absorption cell 113 (i.e., to a minimum operating pressure), gas characterization system 110 reduces molecular interactions which may interfere (i.e., collisional broadening) with gas characterization measurements. Waveguide absorption cell 113 comprises a cell body that defines an optical path through the gas sample. Processing circuitry 111 controls laser 112 to generate an optical beam. Laser 112 generates the optical beam and directs the optical beam through the optical path of waveguide absorption cell. The optical beam generated by laser 112 is typically tuned to wavelengths (i.e., scanned across a range of wavelengths) that correspond to the absorption peaks of the chemicals of interest in the gas sample (i.e., to detect the presence of a particular compound or lack thereof). In waveguide absorption cell 113, the optical beam interacts with the gas sample. For example, the gas sample may absorb a portion of the optical beam emitted from laser 112 in vibrations and rotations of certain molecules. The optical beam exits waveguide absorption cell 113. Photodetector 114 detects the optical beam after its interaction with the gas sample.

Photodetector 114 generates a signal that characterizes the interaction between the optical beam and the gas sample. For example, photodetector 114 may measure the received intensity of the optical beam at different wavelengths and produce a signal that indicates the received intensity for each measured wavelength. For example, the optical beam may be modulated with a high-frequency modulation signal (i.e., wavelength modulation) and photodetector 114 may produce a signal that indicates resulting modulation-induced harmonic components. Photodetector 114 transfers the signal to processing circuitry 111.

Processing circuitry 111 determines the composition of the gas sample based on the received signal. Processing circuitry 111 may use one or more lock-in amplifiers to demodulate the signal and determine the composition of the gas sample based on the demodulated signal. Processing circuitry 111 may control laser 112 to operate in direct absorption measurements and WMS mode. When operating in direct absorption mode, processing circuitry 111 may determine the composition of the gas based on the intensity of the optical beam received at different wavelengths that correspond to absorption peaks of molecules. When operating in WMS mode, processing circuitry 111 may determine patterns in harmonic components (e.g., the second harmonic (2f)) of the signal to determine composition of the gas. These different detection/operating modes give processing circuitry 111 a broad dynamic range. For example, processing circuitry 111 may measure both highly concentrated or highly absorbing components of the gas sample as well as components of gas sample that exist in low-concentrations (e.g., in the parts-per million (ppm), parts-per billion (ppb), or parts-per trillion (ppt) range). Gas characterization system 110 may utilize multiple lasers, multiple photodetectors, multiple lock-in amplifiers, and multiple cell bodies in waveguide absorption cell 113 to enable simultaneous operation of these operating modes. Processing circuitry 111 produces data that indicates the composition of the gas sample as an output.

In some examples, processing circuitry 111 may be coupled with a reaction controller (not illustrated) that controls the reaction rate by modifying the flowrate, composition, or other variables of the reaction inputs delivered to reaction chamber 101. The controller may comprise a Programmable Logic Controller (PLC), a Proportional-Integral-Derivative (PID) controller, a machine learning based controller, and the like. Gas characterization system 110 may indicate the composition of the gas sample to the controller. The controller may adjust the composition, flowrate, and/or other variable of the reaction inputs based on the reported gas composition to achieve a desired gaseous reaction output composition.

Advantageously, gas characterization system 110 efficiently and effectively determines the chemical composition of the gas sample, including detecting trace components of the gas sample. Moreover, gas characterization system 110 may be operated in different detection modes to detect highly concentrated components of the gas sample as well as components of the gas sample in ppm to ppt range.

Processing circuitry 111 may comprise a microcontroller, industrial controller, general-purpose processor, and the like. Processing circuitry 111 may control operation of laser 112, including wavelength tuning and modulation, and may receive, digitize, demodulate, and analyze signals generated by photodetector 114 to determine a composition of the gas sample. Laser 112 may comprise an infrared Distributed Feedback (DFB) laser, External-Cavity Diode Laser (ECDL), Fabry-Pŕot (FP) Diode Laser, Vertical-Cavity Surface-Emitting Laser (VCSEL), Quantum Cascade Laser (QCL), Interband Cascade Laser (ICL), tunable solid-state or fiber laser, and the like. Alternatively, laser 112 may be replaced by or used in addition to a non-laser optical source like a Light Emitting Diode (LED) or lamp.

Waveguide absorption cell 113 may comprise waveguides, light-pipes, reflectors, mirrors, recessed surface features like etchings or machined engravings, and the like to form the optical path. Waveguide absorption cell 113 may comprise a long or short path-length absorption cell. For a short path-length (i.e., substantially equal to or less than a longest cell dimension) absorption cell, the optical path may be substantially linear. For a long path-length (i.e., greater than a longest cell dimension) absorption cell, the optical path may be folded (i.e., a zigzag pattern, a coil pattern, a spiral pattern, etc.). For example, waveguide absorption cell 113 may comprise a monolithic metallic or reflective material (e.g., an aluminum piece) with a double-backing wave path formed (e.g., etched or machined) into the metallic or reflective material to create the folded optical path. For example, waveguide absorption cell 113 may comprise a monolithic metallic or reflective material with a spiraling wave path formed into the metallic or reflective material to create the folded optical path.

Photodetector 114 may comprise a reverse-biased PIN photodiode mated to a fixed gain transimpedance amplifier, avalanche photodiode, photoconductive detector, Schottky photodiode, photovoltaic photodiode, thermopile detector, bolometer, photomultiplier tubes, and the like. The photosensitive material of photodetector 114 may depend on the wavelength of the rotational-vibrational transitions of the gas being detected. Exemplary photosensitive materials include Silicon (Si) for 400-1000 nm, Indium Gallium Arsenide (InGaAs) for 800-1800 nm (which can be extended to 1200-2600 nm), Indium Arsenide Antimonide (InAsSb) for 1000-5300 nm, as well as other photosensitive materials. While illustrated as separate components, processing circuitry 111, laser 112, waveguide absorption cell 113, and photodetector 114 may comprise a single instrument package that is under 10 cm×10 cm×20 cm in size.

Processing circuitry 111, laser 112, and photodetector 114 may communicate over various wireline and/or wireless networking protocols. The communication links comprise metallic links, glass fibers, radio channels, or some other communication media. Processing circuitry 111, laser 112, and photodetector 114 may comprise microprocessors, software, memories, transceivers, bus circuitry, lock-in amplifiers, AD630 chip based lock-in amplifiers, and the like. The microprocessors comprise Central Processing Units (CPUs), Graphical Processing Units (GPUs), Digital Signal Processors (DSPs), Application-Specific Integrated Circuits (ASICs), Field Programmable Gate Arrays (FPGAs), analog computing circuits, and/or the like. The memories comprise Random Access Memory (RAM), flash circuitry, Hard Disk Drives (HDDs), Solid State Drives (SSDs), Non-Volatile Memory Express (NVMe) SSDs, and/or the like. The memories store software like operating systems, gas analysis applications, control applications, and the like. The microprocessors retrieve the software from the memories and execute the software to drive the operation of gas characterization system 110 as described herein.

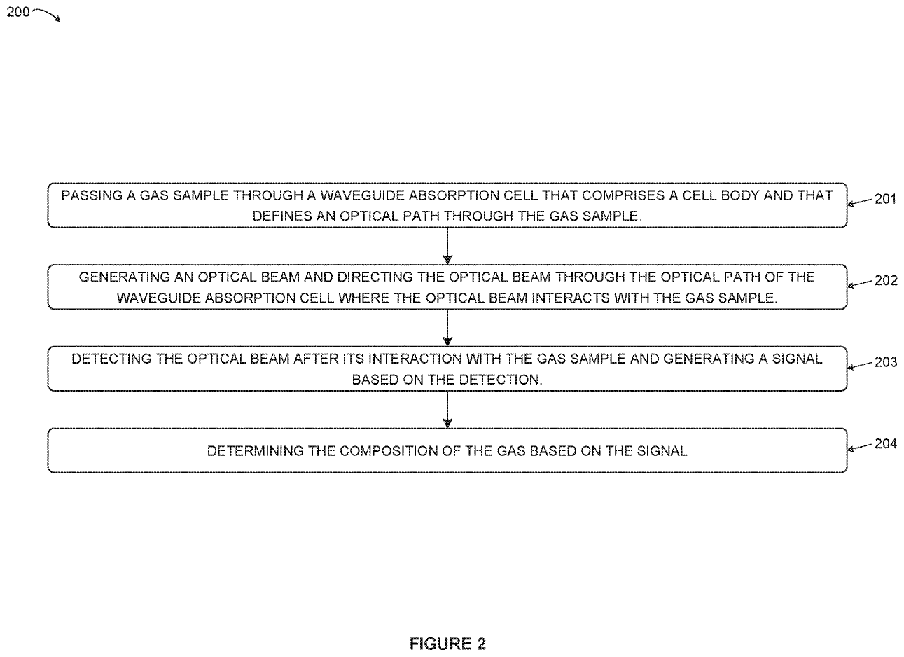

In some examples, gas characterization system 110 implements process 200 illustrated in FIG. 2. It should be appreciated that the structure and operation of gas characterization system 110 may differ in other examples.

FIG. 2 illustrates an example of process 200. Process 200 comprises an example operation of gas characterization system 110 to characterize the composition of a gas. Process 200 may differ in other examples. In some examples, the operations of process 200 comprise passing a gas sample through a waveguide absorption cell that comprises a cell body and that defines an optical path through the gas sample (step 201). The operations further comprise generating an optical beam and directing the optical beam through the optical path of the waveguide absorption cell where the optical beam interacts with the gas sample (step 202). The operations further comprise detecting the optical beam after its interaction with the gas sample and generating a signal based on the detection (step 203). The operations further comprise determining the composition of the gas based on the signal (step 204).

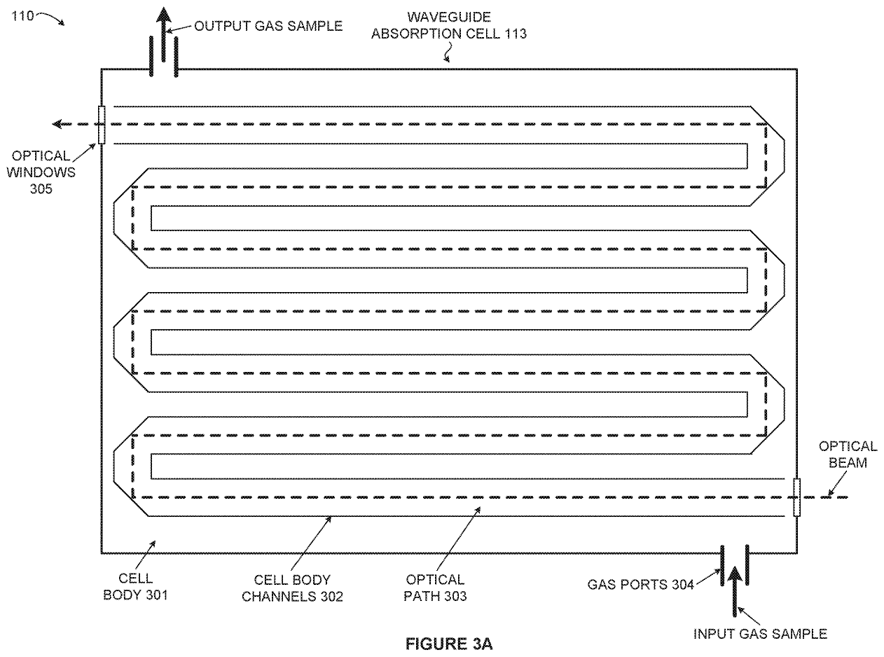

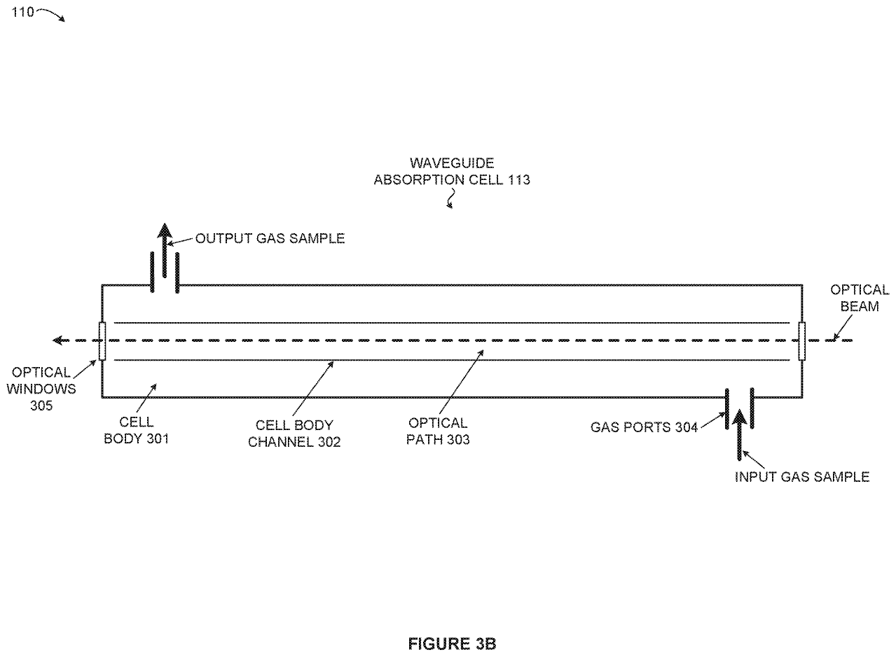

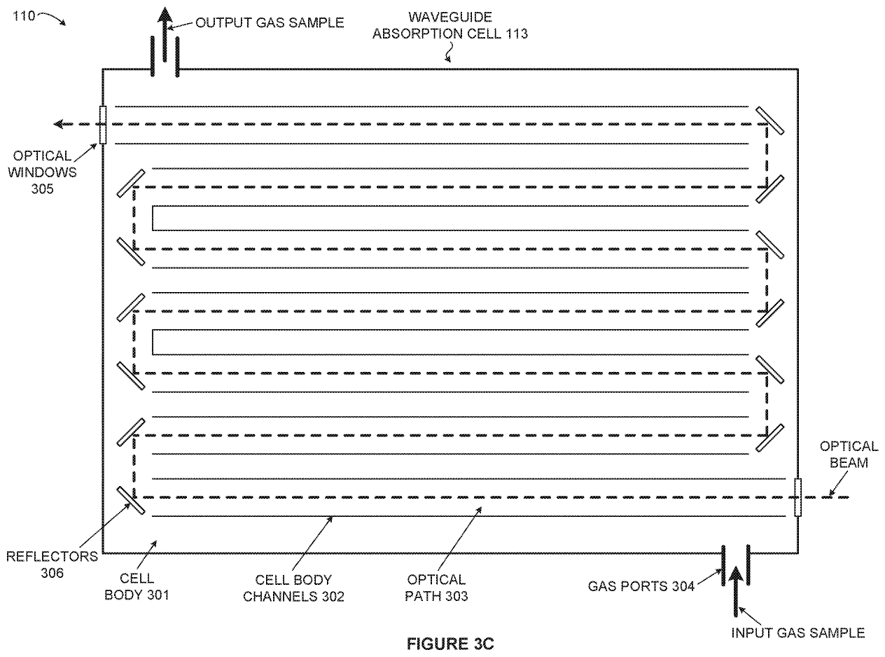

FIG. 3A illustrates an example of waveguide absorption cell 113 in gas characterization system 110 that characterizes the composition of a gas. In some examples, waveguide absorption cell 113 comprises cell body 301, cell body channels 302, optical path 303, gas ports 304, and optical windows 305. Waveguide absorption cell 113 is illustrated in FIG. 3A in a long path-length (i.e., folded path) configuration, however waveguide absorption cell 113 may comprise a short path-length (i.e., substantially linear) configuration. While illustrated as having a double-backing optical path, in other examples the folded path may be a spiral or coil pattern. The long path-length configuration of waveguide absorption cell 113 may be used to measure gas composition in direct absorption mode or WMS mode.

Cell body 301 may comprise a single or multipiece construction with open features (i.e., cell body channels 302) that form optical path 303. For example, cell body 301 may comprise a monolithic solid piece constructed from a reflective material like aluminum, gold, or some other type of reflective material. The reflective material is selected for having sufficient reflectivity to reflect the optical beam through optical path 303. Alternatively, cell body 301 may be constructed from a non-reflective material (e.g., glass, molded plastic, 3D printed plastic, etc.) and have a thin-film deposition of a reflective material (e.g., gold, aluminum, etc.) on its surface. The thin-film deposition may cover the entirety of the non-reflective cell body or may be limited to covering cell body channels 302.

Cell body channels 302 may comprise a waveguide, open space, recessed feature, indentation, light pipe, or other feature machined/etched onto or affixed to cell body 301 forming optical path 303. Cell body channels 302 comprise a set of folded (double-backing) lanes across the width of cell body 301. While the lanes of cell body channels 302 are illustrated as being angled to reflect the optical beam, in other examples cell body channels 302 may be curved. Cell body channels 302 may be created by machining, chemical etching, and/or another process. Alternatively, cell body channels 302 may be created by mounting waveguides to cell body 301. For example, cell body 301 may be constructed from aluminum. Cell body channels 302 may be created by machining the aluminum piece using a CNC milling tool. For example, cell body 301 may be constructed from glass. Cell body channels 302 may be created on the surface using Buffered Oxide Etching (BOE) like Hydrofluoric (HF) acid etching. Aluminum, gold, or another type of reflective material may then be deposited on the surface along cell body channels 302 to create the reflective waveguide. While illustrated as comprising seven double-backing channels, optical path 303 may comprise any number of channels, including one. It should be appreciated that by increasing the number of channels, this increases the length of optical path 303. Increasing path length also increases the sensitivity of waveguide absorption cell 113 in accordance with Beer's Law:

A=ΕLc

where c represents concentration (e.g., the concentration of the chemical component of interest in the gas sample), L represents path length (e.g., length of optical path 303), ϵ represents molar absorptivity (e.g., the absorptivity of the chemical component of interest in the gas sample), and A represents the total absorbance of the gas sample.

Optical windows 305 comprise transparent ports that allow the optical beam to enter optical path 303. Optical windows 305 may be constructed from glass or another type of transparent material that allows the wavelength of interest to enter optical path 303. Gas ports 304 comprise an inlet and outlet that allow gas sample to flow through waveguide absorption cell 113 filling optical path 303. Gas ports 304 may be operatively coupled to or be equipped with a vacuum pump, fan, or other device to create a negative pressure differential to draw the gas sample into cell body 301. Gas ports 304 may also include an adjustable valve to control the flowrate and thereby the pressure within cell body 301. As previously mentioned, reducing the pressure of the sample gas within cell body 301 is desirable to mitigate collisional broadening which may degrade gas characterization measurements.

Advantageously, by utilizing a solid, single-piece or multi-piece reflective body with a wave path (e.g., an etched or machined wave path), waveguide absorption cell 113 may obviate the need to utilize mirrors to reflect the optical beam along folded optical path 303. Eliminating the use of mirrors also eliminates the tedious and difficult process of accurately aligning the mirrors to reflect the laser light along the wave path. This reduces the cost and the complexity to manufacture waveguide absorption cell 113. Although eliminating the use of mirrors may be advantageous, in other examples waveguide absorption cell 113 may utilize mirrors. Moreover, by using a folded optical path longer than a longest dimension of cell body 301, waveguide absorption cell 113 increases its sensitivity in accordance with Beer's Law. This allows waveguide absorption cell 113 to use a lower operating pressure than what would otherwise be possible (e.g., using a short-length or substantially linear wave path). The lower operating pressure increases the quality (i.e., interpretability) of the resulting gas characterization measurements by reducing collisional broadening caused by intermolecular interactions. While waveguide absorption cell 113 is described with respect to passing an optical beam from a laser through cell body channels 302 along optical path 303, other light sources (e.g., non-laser) may be used. For example, the laser may be replaced by an LED or lamp. As such, waveguide absorption cell 113 may be adapted for use with Fourier Transform Infrared Spectroscopy (FTIR) device, infrared gas analyzes, and the like to increase the light path length.

FIG. 3B illustrates another example of waveguide absorption cell 113 in gas characterization system 110 that characterizes the composition of a gas. In some examples, waveguide absorption cell 113 comprises a short path-length (i.e., substantially linear path as opposed to the folded path illustrated in FIG. 3A) configuration as illustrated in FIG. 3B. The short path-length configuration of waveguide absorption cell 113 may be used to measure gas composition in direct absorption mode and may be less commonly used in WMS mode. It should be appreciated that using a short path-length waveguide reduces the sensitivity of the waveguide in accordance with Beer's Law. However, by using a substantially linear waveguide, the construction of waveguide absorption cell 113 is simplified and construction cost is reduced.

FIG. 3C illustrates another example of waveguide absorption cell 113 in gas characterization system 110 that characterizes the composition of a gas. In some examples, waveguide absorption cell 113 further comprises reflectors 306. Waveguide absorption cell 113 is illustrated in FIG. 3C in a long path-length configuration. Reflectors 306 are representative of mirrors or other reflective surfaces to route the optical beam down each lane of cell body channels 302. While using mirrors or other types of reflectors may increase the time, cost, and difficulty in constructing waveguide absorption cell 113, reflectors 306 may, in some embodiments, increase the efficiency at which the optical beam traverses optical path 303 when compared to a continuous etched/machined channel. This may decrease the amount of light that is scattered or otherwise lost while traversing optical path 303 and increase the amount of light that reaches photodetector 114 thereby increasing the overall efficiency of gas composition testing system 113.

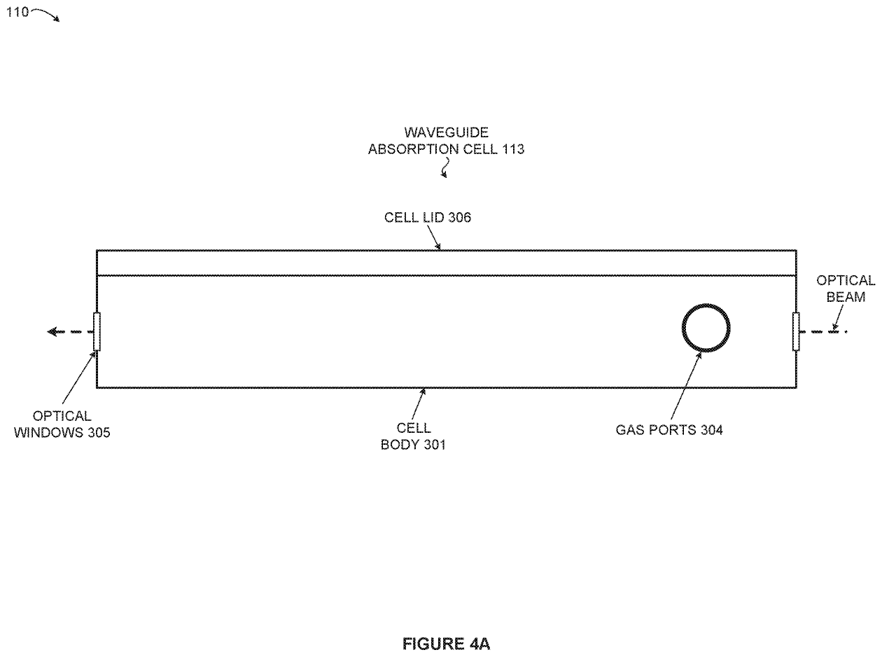

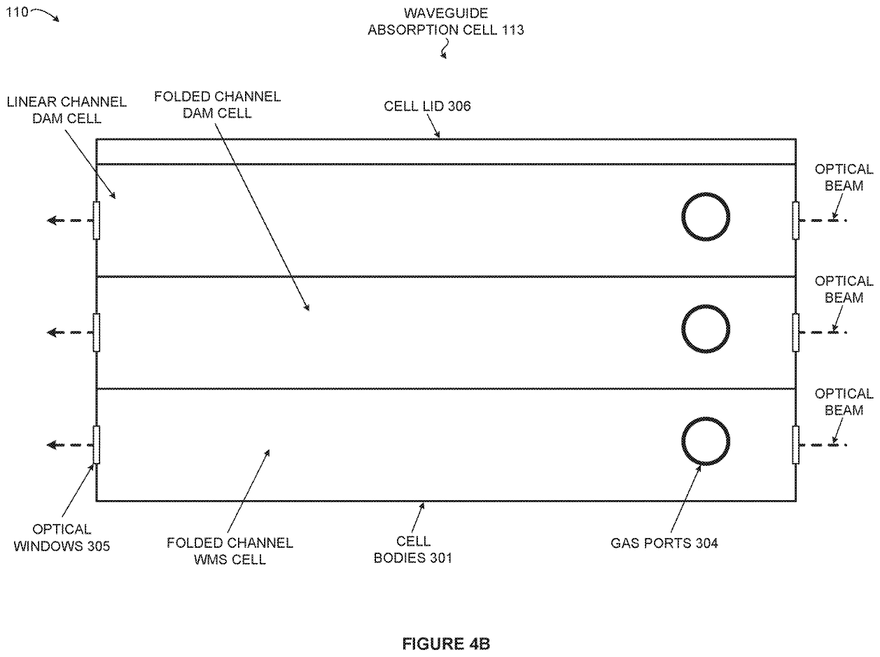

FIG. 4A illustrates an example operating configuration of waveguide absorption cell 113 in gas characterization system 110 that characterizes the composition of a gas. FIG. 4A depicts waveguide absorption cell 113 from a side perspective. In some examples, waveguide absorption cell 113 further comprises cell lid 306. Cell lid 306 is mechanically coupled to the top of cell body 301 (i.e., the side of cell body 301 with cell body channels 302). Cell lid 306 forms a seal that prevents or otherwise inhibits the gas sample from leaking out of waveguide absorption cell 113 or gas from the environment leaking into waveguide absorption cell 507.

FIG. 4B illustrates an example of an alternate operating configuration of waveguide absorption cell 113 in gas characterization system 110 that characterizes the composition of a gas. FIG. 4B depicts waveguide absorption cell 113 from a side perspective. In some examples, waveguide absorption cell 113 comprises multiple cell bodies that correspond to different operating modes. This allows gas characterization system 110 to simultaneously operate in direct absorption mode (DAM) and WMS mode. As illustrated in FIG. 4B, the top one of cell bodies 301 comprises a linear channel direct absorption mode cell (i.e., the configuration of waveguide absorption cell 113 illustrated in FIG. 3B), the middle one of cell bodies comprises a folded channel direct absorption mode cell (i.e., the configuration of waveguide absorption cell 113 illustrated in either FIG. 3A or 3C), and the bottom one of cell bodies comprises a folded WMS mode cell (i.e., the configuration of waveguide absorption cell 113 illustrated in either FIG. 3A or 3C). The pathlength for the WMS mode cell is typically equal to or greater than the folded channel direct absorption mode cell. For example, the linear channel direct absorption mode cell may be used to measure high concentration components, the folded channel direct absorption mode cell may be used to measure mid-concentration components, and the folded channel WMS mode cell may be used to measure low-concentration (e.g., ppm or ppt) components. Each of cell bodies 301 may be operatively coupled with a corresponding photodetector which measures and reports the resulting signal to processing circuitry 111. This multicell configuration enables gas characterization system 110 to achieve a large dynamic detection range by combining direct absorption measurements, WMS measurements, and a range of waveguide absorption cell path lengths.

FIG. 5 illustrates an example of gas composition testing system 500. Gas composition testing system 500 comprises an example of gas characterization system 110 illustrated in FIG. 1, however gas characterization system 110 may differ. In some examples, gas composition testing system 500 comprises processing circuitry 501, laser controller 502, laser 503, fiber switch 504, fiber optic 505, waveguide absorption cell 507, photodiode 508, and lock-in amplifier 509. In other examples, gas composition testing system 500 may include fewer or additional components than those illustrated in FIG. 5. Likewise, the illustrated components of gas composition testing system 500 may include fewer or additional components, assets, or connections than shown.

In some examples, gas composition testing system 500 is representative of a Tunable Diode Laser Absorption Spectroscopy (TDLAS) system or apparatus to measure trace gases in natural gas combustion systems (or other types of gas producing processes). Gas composition testing system 500 is illustrated in a direct absorption mode configuration in FIG. 5 but may operate in WMS mode which is depicted in FIG. 6. Combustion exhaust resulting from the burning of natural gas (or other hydrocarbon combustion reaction) is sampled and passed to waveguide absorption cell 507 (e.g., a monolithic aluminum piece with a folded wave path etched/machined into the aluminum piece) as an input. The gas flows through waveguide absorption cell 507 and exits waveguide absorption cell 507 as an output. As the sampled exhaust is flowing through waveguide absorption cell 507, processing circuitry 501 directs laser controller 502 to activate laser 503 (e.g., an infrared DFB laser). Laser controller activates laser 503. Laser 503 transfers a laser beam through fiber switch 504, fiber optic 505, and optics 506 to waveguide absorption cell 507. Fiber switch 504 modulates the laser beam and sends an electronic reference signal to lock-in amplifier 509 for demodulation. Optics 506 collimates the laser beam. In waveguide absorption cell 507, the flowing gas absorbs a portion of the laser beam emitted from laser 503 in vibrations and rotations of certain molecules. After passing through waveguide absorption cell 507, the laser beam is detected by photodiode 508 (e.g., a reverse-biased PIN photodiode mated to a fixed gain transimpedance amplifier).

Photodiode 508 measures the intensity of the laser beam after interacting with the gas sample in waveguide absorption cell 507. The beam generated by laser 503 is tuned to wavelengths that correspond to the absorption peaks of methane, carbon monoxide, nitrous oxide, or other pollutants/chemicals of interest resulting from the incomplete combustion of natural gas or from undesired side reactions. Laser controller 502 interfaces with laser 503 to adjust the wavelength of the laser beam to scan across the wavelengths of interest (e.g., by adjusting the operating temperature of laser 503). For example, when configured to detect methane, laser 503 may produce a laser beam between the wavelengths 1640.2 and 1640.5 nm to capture methane absorption features. Photodiode 508 produces a signal based on the detected laser beam and transfers the signal to lock-in amplifier 509 (e.g., an AD630 chip based lock-in amplifier). The signal characterizes the frequency, phase, wavelength, harmonic components, and/or some other aspect of the laser beam after interacting with the gas sample.

Lock-in amplifier 509 processes the signal from photodiode 508 based on the reference signal. The signal from the photodiode is amplified by lock-in amplifier 509 and referenced to the modulation frequency of fiber switch 504. The output of lock-in amplifier 509 identifies the amount of absorbed laser light at the absorption peaks for the chemical components (i.e., methane, carbon monoxide, nitric oxide, etc.) of the sampled exhaust. Lock-in amplifier 509 converts the analog output into a digital signal and provides the digital signal to processing circuitry 501 (e.g., a microcontroller with an analog-to-digital converter and DSP). As laser 503 scans across a wavelength of interest, molecules in the gas sample absorb light from through their vibrations and rotations. These wavelengths of absorption are known for each molecule and are referred to as “lines” or “absorption features.” The depth of these lines or absorption features can be correlated with the mole fraction (concentration) of the gas in the sample. Scanning the laser beam across the lines (absorption features) and detecting the output produces an absorption spectra. Processing circuitry 501 determines the composition of the gas sample based on the peaks of the absorption spectra. Processing circuitry 501 adjusts the voltages supplied to laser 503 (via laser controller 502) to modify the wavelength of the beam to sense a new wavelength.

FIG. 6 illustrates another example of gas composition testing system 500. In some examples, gas composition testing system 500 further comprises lock-in amplifier 510. Gas composition testing system 500 is illustrated in a WMS operating mode in FIG. 6 as opposed to the direct absorption mode depicted in FIG. 5. When operating in WMS mode, gas composition testing system operates similarly as described above with respect to FIG. 5, however by utilizing multiple lock-in amplifiers, gas composition testing system 500 increases its detection sensitivity. For WMS, two lock-in amplifiers are typically used. The signal from photodiode 508 is split and separately amplified by lock-in amplifiers 509 and 510. Lock-in amplifier 510 is referenced to a high frequency sinusoidal modulation applied to the laser current internally through laser controller 502. The modulated laser beam interacts with the sampled exhaust in waveguide absorption cell 507. Absorption lines impose distinctive non-linear harmonic patterns on the modulated signal. Photodiode 508 detects the beam and passes a resulting analog signal to lock-in amplifiers 509 and 510. Lock-in amplifier 510 demodulates the second harmonic “2f” signal which is proportional to the absorption derivative. Lock-in amplifier 509 is referenced to the fiber switch modulation frequency which provides a measure of laser power. The 2f spectra is generated after laser 503 scans in wavelength across the rotational-vibration transitions (absorption features). The outputs of lock-in amplifiers 509 and 510 are passed to processing circuitry 501. Processing circuitry 501 determines the composition of the sampled exhaust gas based on the magnitude and shape of the 2f harmonic component of the demodulated signal.

Advantageously, gas composition testing system 500 efficiently and effectively determines the chemical composition of the gas sample, including detecting trace components of the gas sample. Moreover, gas composition testing system 500 is multimodal using both direct absorption and WMS as well as waveguide absorption cells with variable cell path lengths to improve its detection range when compared to conventional systems. For example, gas composition testing range 500 may detect trace pollutants (i.e., the ppm, ppb, or ppt range) in exhaust gas. Furthermore, gas composition testing system 500 may be coupled with a combustion controller to adjust fuel/air ratios during combustion based on the determined gas composition. By adjusting the fuel/air ratio based on exhaust gas composition, gas composition testing system 500 may be used to limit the amount of unreacted combustion inputs and/or undesired reaction side products that are expelled into the atmosphere. This reduces the number of pollutants (e.g., carbon monoxide (CO), nitric oxides (NO_{X}), hydrogen sulfide (H₂S), and sulfur oxides (SO_{X}), etc.) and harmful greenhouse gases (e.g., natural gas, methane (CH₄), ethane (C₂H₆), propane (C₃H₈), and butane (C₄H₁₀), etc.) that are released into the atmosphere. Monitoring gas compositions in tandem with combustion system inputs may be used to optimize combustion processes by reducing products of incomplete combustion and reducing the amount of greenhouse gases that result from non-optimal burn conditions.

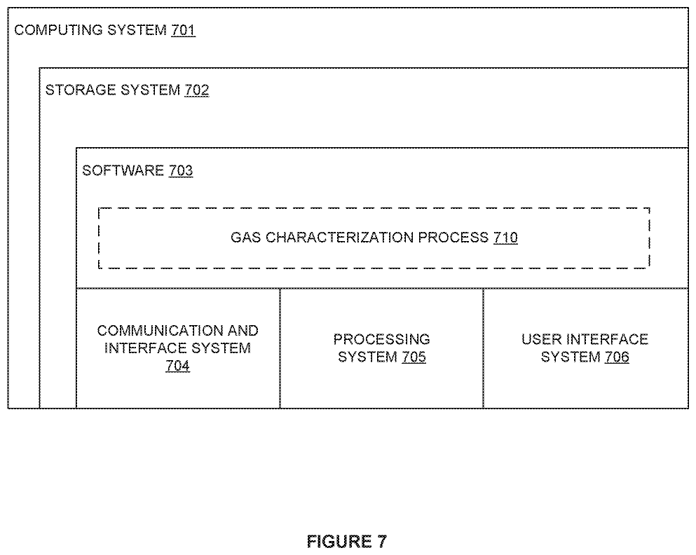

FIG. 7 illustrates computing system 701. Computing system 701 is representative of any system or collection of systems with which the various operational architectures, processes, scenarios, and sequences disclosed herein for collecting and characterizing the chemical composition of gas. For example, computing system 701 may be representative of processing circuitry 501, laser controller 502, lock-in amplifiers 109 and 110, and/or any other computing device contemplated herein. Computing system 701 may be implemented as a single apparatus, system, or device or may be implemented in a distributed manner as multiple apparatuses, systems, or devices. Computing system 701 includes, but is not limited to, storage system 702, software 703, communication interface system 704, processing system 705, and user interface system 706. Processing system 705 is operatively coupled with storage system 702, communication interface system 704, and user interface system 706.

Processing system 705 loads and executes software 703 from storage system 702. Software 703 includes and implements gas characterization process 710, which is representative of any of the gas characterization processes described with respect to the preceding Figures, including but not limited to the gas sampling, gas characterization, and control operations described with respect to the preceding Figures. For example, process 710 may be representative of process 200 illustrated in FIG. 2. When executed by processing system 705 to sample and characterize gas, software 703 directs processing system 705 to operate as described herein for at least the various processes, operational scenarios, and sequences discussed in the foregoing implementations. Computing system 701 may optionally include additional devices, features, or functionality not discussed for purposes of brevity.

Processing system 705 may comprise a micro-processor and other circuitry that retrieves and executes software 703 from storage system 702. Processing system 705 may be implemented within a single processing device but may also be distributed across multiple processing devices or sub-systems that cooperate in executing program instructions. Examples of processing system 705 include general purpose CPUs, GPUs, DSPs, ASICs, FPGAs, analog computing devices, and logic devices, as well as any other type of processing device, combinations, or variations thereof.

Storage system 702 may comprise any computer readable storage media readable by processing system 705 and capable of storing software 703. Storage system 702 may include volatile, nonvolatile, removable, and/or non-removable media implemented in any method or technology for storage of information, such as computer readable instructions, data structures, program modules, or other data. Examples of storage media include RAM, read only memory, magnetic disks, optical disks, optical media, flash memory, virtual memory and non-virtual memory, magnetic cassettes, magnetic tape, magnetic disk storage or other magnetic storage devices, or any other suitable storage media. In no case is the computer readable storage media a propagated signal.

In addition to computer readable storage media, in some implementations storage system 702 may also include computer readable communication media over which at least some of software 703 may be communicated internally or externally. Storage system 702 may be implemented as a single storage device but may also be implemented across multiple storage devices or sub-systems co-located or distributed relative to each other. Storage system 702 may comprise additional elements, such as a controller, capable of communicating with processing system 705 or possibly other systems.

Software 703 (including process 710) may be implemented in program instructions and among other functions may, when executed by processing system 705, direct processing system 705 to operate as described with respect to the various operational scenarios, sequences, and processes illustrated herein. For example, software 703 may include program instructions for generating characterization exhaust gas composition based on photodiode signals as described herein.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel, in a single threaded environment or multi-threaded, or in accordance with any other suitable execution paradigm, variation, or combination thereof. Software 703 may include additional processes, programs, or components, such as operating system software, virtualization software, or other application software. Software 703 may also comprise firmware or some other form of machine-readable processing instructions executable by processing system 705.

In general, software 703 may, when loaded into processing system 705 and executed, transform a suitable apparatus, system, or device (of which computing system 701 is representative) overall from a general-purpose computing system into a special-purpose computing system customized to sample and characterize gas. Indeed, encoding software 703 on storage system 702 may transform the physical structure of storage system 702. The specific transformation of the physical structure may depend on various factors in different implementations of this description. Examples of such factors may include, but are not limited to, the technology used to implement the storage media of storage system 702 and whether the computer-storage media are characterized as primary or secondary storage, as well as other factors.

For example, if the computer readable storage media are implemented as semiconductor-based memory, software 703 may transform the physical state of the semiconductor memory when the program instructions are encoded therein, such as by transforming the state of transistors, capacitors, or other discrete circuit elements constituting the semiconductor memory. A similar transformation may occur with respect to magnetic or optical media. Other transformations of physical media are possible without departing from the scope of the present description, with the foregoing examples provided only to facilitate the present discussion.

Communication interface system 704 may include communication connections and devices that allow for communication with other computing systems (not shown) over communication networks (not shown). Examples of connections and devices that together allow for inter-system communication may include network interface cards, antennas, power amplifiers, radiofrequency circuitry, transceivers, and other communication circuitry. The connections and devices may communicate over communication media to exchange communications with other computing systems or networks of systems, such as metal, glass, air, or any other suitable communication media. The aforementioned media, connections, and devices are well known and need not be discussed at length here.

Communication between computing system 701 and other computing systems (not shown), may occur over a communication network or networks and in accordance with various communication protocols, combinations of protocols, or variations thereof. Examples include intranets, internets, the Internet, local area networks, wide area networks, wireless networks, wired networks, virtual networks, software defined networks, data center buses and backplanes, or any other type of network, combination of networks, or variation thereof. The aforementioned communication networks and protocols are well known and an extended discussion of them is omitted for the sake of brevity.

While some examples provided herein are described in the context of computing devices for gas characterization, it should be understood that the condition systems and methods described herein are not limited to such embodiments and may apply to a variety of other environments and their associated systems. As will be appreciated by one skilled in the art, aspects of the present invention may be embodied as a system, method, computer program product, and other configurable systems. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

These and other changes can be made to the technology in light of the above Detailed Description. While the above description describes certain examples of the technology, and describes the best mode contemplated, no matter how detailed the above appears in text, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the above Detailed Description section explicitly defines such terms. Accordingly, the actual scope of the technology encompasses not only the disclosed examples, but also all equivalent ways of practicing or implementing the technology under the claims

What is claimed is:

1. A gas characterization system comprising:
a waveguide absorption cell, processing circuitry, a laser, and a photodetector;
the waveguide absorption cell configured to receive a gas sample, wherein the waveguide absorption cell comprises a cell body that defines an optical path through the gas sample;
the laser configured to generate an optical beam and direct the optical beam through the optical path of the waveguide absorption cell, wherein the optical beam interacts with the gas sample;
the photodetector configured to detect the optical beam after its interaction with the gas sample and generate a signal based on the detection; and
the processing circuitry configured to determine a composition of the gas sample based on the signal.

2. The gas characterization system of claim 1, further comprising at least one lock-in amplifier configured to demodulate the signal with respect to a reference modulation signal, wherein the processing circuitry is configured to determine the composition of the gas sample based on the demodulated signal.

3. The gas characterization system of claim 1 wherein:
the processing circuitry is further configured to control the laser to operate in direct absorption mode by adjusting a wavelength of the optical beam to scan across a target absorption spectra;
the photodetector is configured to detect the optical beam after its interaction with the gas sample and generate the signal that indicates transmitted intensities at scanned wavelengths; and
the processing circuitry is configured to determine the composition of the gas sample based on the transmitted intensities at the scanned wavelengths.

4. The gas characterization system of claim 1 wherein:
the processing circuitry is further configured to control the laser to operate in Wavelength Modulation Spectroscopy (WMS) mode by adjusting a wavelength of the optical beam to scan across a target absorption spectra and simultaneously modulating the optical beam with a high-frequency modulation signal; and
the photodetector is configured to detect the optical beam after its interaction with the gas sample and generate the signal that indicates modulation-induced harmonic components; and
the processing circuitry is further configured to determine the composition of the gas sample based on the modulation-induced harmonic components.

5. The gas characterization system of claim 4, further comprising at least one lock-in amplifier configured to demodulate the modulation-induced harmonic components and provide demodulated harmonic components to the processing circuitry, wherein the processing circuitry is configured to determine the composition of the gas sample based on the demodulated harmonic components.

6. The gas characterization system of claim 1 wherein the cell body of the waveguide absorption cell comprises a single waveguide that defines a substantially linear optical path.

7. The gas characterization system of claim 1 wherein the cell body of the waveguide absorption cell comprises multiple waveguides and multiple reflectors that define a folded optical path.

8. The gas characterization system of claim 1 wherein the cell body of the waveguide absorption cell comprises a reflective channel that defines a folded optical path.

9. A method comprising:
passing a gas sample through a waveguide absorption cell, wherein the waveguide absorption cell comprises a cell body that defines an optical path through the gas sample;
generating an optical beam and directing the optical beam through the optical path of the waveguide absorption cell, wherein the optical beam interacts with the gas sample;
detecting the optical beam after its interaction with the gas sample and generating a signal based on the detection; and
determining a composition of the gas sample based on the signal.

10. The method of claim 9 further comprising demodulating the signal with respect to a reference modulation signal, wherein determining the composition of the gas sample based on the signal comprises determining the composition of the gas sample based on the demodulated signal.

11. The method of claim 9 further comprising adjusting a wavelength of the optical beam to scan across a target absorption spectra to operate in direct absorption mode, wherein:
detecting the optical beam after its interaction with the gas sample and generating the signal based on the detection comprises detecting the optical beam after its interaction with the gas sample and generating the signal that indicates transmitted intensities at scanned wavelengths; and
determining the composition of the gas sample based on the signal comprises determining the composition of the gas sample based on the transmitted intensities at the scanned wavelengths.

12. The method of claim 9 further comprising adjusting a wavelength of the optical beam to scan across a target absorption spectra and simultaneously modulating the optical beam with a high-frequency modulation signal to operate in Wavelength Modulation Spectroscopy (WMS) mode, wherein:
detecting the optical beam after its interaction with the gas sample and generating the signal based on the detection comprises detecting the optical beam after its interaction with the gas sample and generating the signal that indicates modulation-induced harmonic components; and
determining the composition of the gas sample based on the signal comprises determining the composition of the gas sample based on the modulation-induced harmonic components.

13. The method of claim 12 further comprising demodulating the modulation-induced harmonic components to produce demodulated harmonic components, wherein determining the composition of the gas sample based on the modulation-induced harmonic components comprises determining the composition of the gas sample based on the demodulated harmonic components.

14. The method of claim 9 wherein the cell body of the waveguide absorption cell comprises a single waveguide that defines a substantially linear optical path.

15. The method of claim 9 wherein the cell body of the waveguide absorption cell comprises multiple waveguides and multiple reflectors that define a folded optical path.

16. The method of claim 9 wherein the cell body of the waveguide absorption cell comprises a reflective channel that defines a folded optical path.

17. A waveguide absorption cell comprising:
a cell body, one or more optical windows, and at least one gas port;
the cell body having one or more open features that form a folded optical path configured to increase an interaction between a gas sample and an optical beam, wherein a total length of the folded optical path is greater than a largest dimension of the cell body;
the one or more optical windows configured to allow the optical beam to pass through the folded optical path; and
the at least one gas port configured to allow the gas sample to flow through the folded optical path.

18. The waveguide absorption cell of claim 17 wherein:
the cell body comprises a reflective surface; and
the one or more open features comprise one or more recessed features in the reflective surface that form the folded optical path.

19. The waveguide absorption cell of claim 17 further comprising one or more reflectors, and wherein:
the one or more open features comprise one or more light pipes; and
the one or more reflectors and the one or more light pipes form the folded optical path.

20. The waveguide absorption cell of claim 17 further comprising a cell lid operatively coupled to the cell body to enclose a folded recessed channel, wherein:
the cell body comprises a monolithic reflective piece;
the one or more open spaces comprise the folded recessed channel etched or machined into the monolithic reflective piece that forms the folded optical path.
